(19) United States
(12) Patent Application Publication
(10) Pub. No.: US 2024/0342347 A1
(43) Pub. Date: Oct. 17, 2024

(54) BIOFOULING RESISTANT COATINGS FOR MEDICAL DEVICES

(71) Applicant: Surmodics, Inc., Eden Prairie, MN (US)

(72) Inventor: Syed Hossainy, Hayward, CA (US)

(21) Appl. No.: 18/612,034

(22) Filed: Mar. 21, 2024

Related U.S. Application Data
(60) Provisional application No. 63/453,575, filed on Mar. 21, 2023.

(51) Int. Cl. A61L 33/00; A61L 29/08; A61L 29/16; A61L 33/06

(52) U.S. Cl. CPC A61L 33/0023; A61L 29/085; A61L 29/16; A61L 33/0064; A61L 33/062; A61L 2300/42

(57) ABSTRACT

Embodiments herein relate to biofouling resistant coatings for medical devices. In an embodiment, an anti-fouling coated medical device is included having a substrate and a coating disposed over the substrate. The coating can include a hydrogel, and a polyzwitterion. The polyzwitterion can include a bound portion and an unbound portion. In some embodiments the coating can further include a heparin compound. Other embodiments are also included herein.

This application claims the benefit of U.S. Provisional Application No. 63/453,575, filed Mar. 21, 2023, the content of which is herein incorporated by reference in its entirety.

### FIELD

Embodiments herein relate to medical device coatings. More specifically, embodiments herein relate to biofouling resistant coatings for medical devices.

### BACKGROUND

Medical devices include those that are chronically implanted, devices that are transitorily implanted, and those that not implanted at all. While necessary and beneficial for treating a variety of medical conditions, implanting medical devices can result in the initiation of a response resulting in biofouling of the surfaces of the medical device, inflammation and fibrous encapsulation of the medical device, and/or the initiation of a wound healing response which can result in hyperplasia. Such effects can negative impacts on the patient and substantially hinder device functionality and/or reduce device lifetime.

### SUMMARY

Embodiments herein relate to biofouling resistant coatings for medical devices. In an embodiment, an anti-fouling coated medical device is included having a substrate and a coating, wherein the coating is disposed on the substrate. The coating can include a hydrogel and a polyzwitterion. The polyzwitterion can include a bound portion and an unbound portion. In some embodiments, the coating can include a heparin compound.

In an embodiment, an anti-fouling coated medical device is included having a substrate and a coating, wherein the coating is disposed on the substrate. The coating can include a hydrogel and a polyzwitterion. The polyzwitterion can include a bound portion and an unbound portion.

In an embodiment, a method of making an anti-fouling coated medical device is included. The method can include depositing a coating on a substrate. The coating can include a hydrogel and a polyzwitterion. The polyzwitterion can include a bound (such as cross-linked) portion and an unbound portion. In some embodiments the coating can include a heparin compound.

In an embodiment, a coated medical device is included having a substrate and a coating disposed thereon. The coating can include a degradable polymer. The degradable polymer can include a polymeric backbone with degradable linkages and polymeric chains grafted onto the backbone. The chains can include a polyzwitterionic polymer, and polyethylene glycol, or a copolymer thereof.

In an embodiment, a coated medical device is included having a substrate and a coating disposed thereon. The coating can include a degradable polymer layer. The degradable polymer layer can include a multi-block copolymer with subunits selected from the group consisting of lactide, glycolide, e-caprolactone, and polyethylene glycol. The coating can include a top layer. The top layer can include a photo-PVP, a heparin compound, and a polyacrylamide polymer.

In an embodiment, a coated medical device is included having a substrate and a coating disposed thereon. The coating can include a degradable polymer. The degradable polymer can include a polymeric backbone with degradable linkages. The polymeric backbone can include a multi-block copolymer with subunits selected from the group consisting of lactide, glycolide, e-caprolactone, and polyethylene glycol, and subunits including at least one selected from the group consisting of sebacic acid and glycerol.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a coated medical device in accordance with various embodiments herein.

FIG. 2 is a cross-sectional view of a coating in accordance with various embodiments herein.

FIG. 3 is a cross-sectional view of a coating in accordance with various embodiments herein.

FIG. 4 is a cross-sectional view of a portion of a coating in accordance with various embodiments herein.

FIG. 5 is a cross-sectional view of a portion of a coating in accordance with various embodiments herein.

FIG. 6 is a graph showing percent retained heparin activity of different coating compositions.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, implanting medical devices can result in the initiation of a response resulting in biofouling of the surfaces of the medical device, inflammation and fibrous encapsulation of the medical device, and/or the initiation of a wound healing response resulting in hyperplasia. However, embodiments herein include medical device coatings and medical devices formed with the same that are effective to reduce such negative effects.

For example, an anti-fouling coated medical device is included herein having a substrate and a coating disposed over the substrate. The coating can include a hydrogel, and a polyzwitterion. The polyzwitterion can include a cross-linked portion and an unbound portion. The coating can further include a heparin compound. While not intending to be bound by theory, when implanted in the body, the unbound portion of the polyzwitterion can migrate out of the coating at least in part forming a polyzwitterion layer on top of the coating. This structure can be highly effective for preventing fouling of the surface.

Many different medical devices can be coated with coatings herein including, but not limited to, chronically implantable medical devices, transitorily implantable medical devices, and the like. Further examples of implantable medical devices are provided below. However, in various embodiments, catheters of various types and/or devices having shafts can be coated.

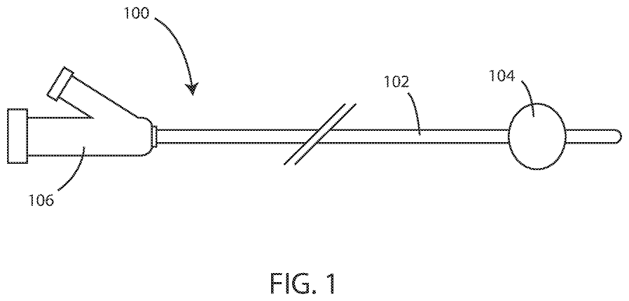

Referring now to FIG. 1, a schematic view of a coated medical device 100 is shown in accordance with various embodiments herein. It will be appreciated that the coated medical device 100 of FIG. 1 is merely one example of a medical device can be coated with coatings described herein. In this example, the coated medical device 100 can include a catheter shaft 102, a balloon 104, and a connection manifold 106 (or proximal connector). The balloon 104 can be inflated and, in some embodiments, can also carry a drug-eluting coating. In various embodiments, the catheter shaft 102 can be coated with a coating herein. In some embodiments, the balloon 104 can be coated with a coating herein. In some embodiments, the catheter shaft 102 and the balloon 104 can be coated with a coating herein.

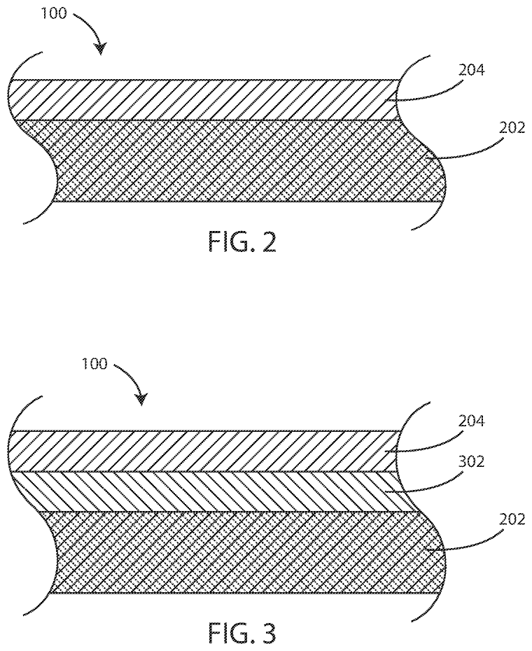

Referring now to FIG. 2, a cross-sectional view of a coating is shown in accordance with various embodiments herein. FIG. 2 shows a coated medical device 100. The coated medical device 100 includes a substrate 202. Example of substrates can include polymers, metals, ceramics, composites, and the like. In various embodiments, the substrate 202 can be at least one selected from the group consisting of a polymer and a metal. Further examples of substrates are provided below. In this example, the coated medical device 100 includes a first layer 204. The first layer 204 can be disposed over the substrate 202. In some embodiments, the first layer 204 can be disposed directly on the substrate 202. In some embodiments, an intermediate layer can be disposed between the first layer 204 and the substrate 202.

In some embodiments, the first layer 204 can be the outer most portion of the coating such that an outer surface of the first layer 204 can configured for direct contact with the in vivo environment into which the coated medical device 100 is inserted. However, in some embodiments another layer (not shown in this view) can be disposed over the first layer 204.

Referring now to FIG. 3, a cross-sectional view of a coating is shown in accordance with various embodiments herein. FIG. 2 is generally similar to FIG. 1 and shows a coated medical device 100. However, in this embodiment, the coated medical device 100 also includes a second layer 302. In this case, the second layer 302 can be disposed under the first layer 204. However, in some embodiments, a second layer 302 can be disposed over the first layer 204. In some embodiments, the second layer 302 can be in direct contact with the first layer 204. However, in some embodiments, an intermediate layer can be disposed between the first layer 204 and the second layer 302. The first layer 204 and the second layer 302 can be composed of the same materials and/or can be composed of different materials.

The first layer 204 can be retained on the second layer 206 in various ways. For example, in some embodiments, the first layer 204 can be retained on the second layer 206 by hydrogen bonding, ionic bonding, covalent bonding, molecular entanglement, or the like.

The components within the first layer 204 and/or the second layer 206 can vary and can include various specific compounds herein. In an embodiment, a coating herein can include a hydrogel and a polyzwitterion.

The polyzwitterion can include a bound portion (such as a cross-linked portion) and an unbound portion. The bound portion can remain bound to the hydrogel after exposure to an aqueous environment, while the unbound portion can elute off. In some embodiments, the ratio of the first portion and the second portion is from 1:99 to 99:1 by weight. In some embodiments, at least about 40, 50, 60, 70, 80, 90, 95, or 99% by weight of the unbound portion elutes off the coating within 1, 2, 3, 4, 5, 6, 7, or 8 weeks of implantation in vivo. In some embodiments, less than 60, 50, 40, 30, 20, or 10% by weight of the unbound portion elutes off the coating within 24, 48, 72, or 96 hours of implantation.

The coating can further include a heparin compound. However, in some embodiments, the coating lacks a heparin compound. Details regarding exemplary hydrogels, polyzwitterions, and heparin compounds can be found below.

In some embodiments, coating layers herein can be formed by applying a coating composition onto a substrate or an underlying layer. For example, a first layer can be formed by applying a first coating composition onto a substrate and, where a second layer is included, a second layer can be formed by applying a second coating composition. In some cases, the layers can be dried or otherwise cured after application and/or prior to applying an overlying layer. In some cases, such as where one or more components includes a photoreactive compound, actinic radiation such as UV light can be applied to the layer or layers to trigger crosslinking and/or polymerization.

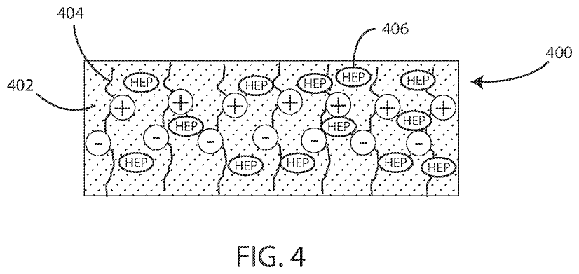

Referring now to FIG. 4, a cross-sectional view of a portion of coating is shown in accordance with various embodiments herein. FIG. 4 shows a layer 400 of a coated medical device 100. The layer 400 (which could be a first layer, a second layer, etc.) can include a hydrogel 402, a polyzwitterion 404, and a heparin compound 406.

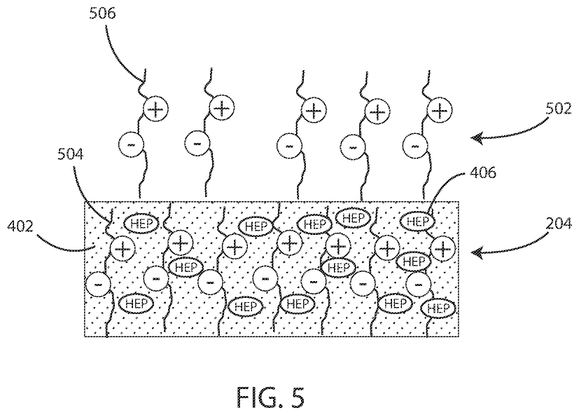

In some cases, a portion of the polyzwitterion can be bound into the hydrogel, such as being covalently bonded thereto. In some cases, a portion of the polyzwitterion can be crosslinked so as to be retained within the hydrogel. However, a portion of the polyzwitterion can be configured so that it migrates out of the hydrogel after insertion into a patient. As such, the polyzwitterion can also include a portion that is unbound. Referring now to FIG. 5, a cross-sectional view of a portion of coating is shown in accordance with various embodiments herein. FIG. 5 is generally similar to FIG. 4. However, in FIG. 5 a portion 506 of the polyzwitterion has migrated out of the hydrogel 402, while another portion 504 remains behind. As such, the portion 506 of the polyzwitterion that has migrated out can form a new surface layer 502 in vivo. In various embodiments, the unbound portion of the polyzwitterion can represent at least about 5, 10, 15, 20, 25, 30, 40, 50, 60, 70, 80, 90, or 95 percent by weight (or an amount falling within a range between any of the foregoing as bounds of the range) of total amount of the polyzwitterion with be balance being the bound portion. In some embodiments, the polyzwitterion is the same compound between the bound and unbound portions. However, in some embodiments, the bound portion may be one type of polyzwitterion compound while the unbound portion may be another type of polyzwitterion compound.

### Methods

Many different methods are contemplated herein, including, but not limited to, methods of making, methods of using, and the like. Specifically, in some embodiments a method of making a coated medical device is included herein. The method can include obtaining a substrate. The method can further include applying a coating over the substrate. The coating can a hydrogel and a polyzwitterion. The polyzwitterion can include a cross-linked portion and an unbound portion. The coating can further include a heparin compound. However, in some embodiments, the coating lacks a heparin compound.

Coating can be performed using various techniques including, but not limited to, spray coating, dip coating, blade coating, printing, contact coating techniques, and the like. Some methods herein can include one or more operations of drying or otherwise curing. Some methods herein can include one or more operations of applying actinic radiation, such as UV light, to the applied layer or layers.

### Hydrogels

Hydrogels are hydrophilic polymers, with a three-dimensional network structure that has the ability to absorb a large volume of water due to the presence of hydrophilic moieties. Hydrogels herein can include both natural and synthetic hydrogels. Hydrogels herein can those that are homopolymers, copolymers, semi-interpenetrating networks, and interpenetrating networks. Hydrogels herein can include those with a cross-linked or uncrosslinked network structure. Hydrogels herein can include those that are charged (cationic and/or anionic groups) and those that are uncharged. Hydrogels herein can include those that are amorphous, semicrystalline, crystalline, and those that are hydrocolloid aggregates.

Hydrogels herein can include, but are not limited to, homopolymer or copolymers of polyethylene glycol (PEG), PEG-PEGMA, polyacrylic acid (PAA), methacrylic acid (MAA), polyethylene oxide (PEO), polyvinyl alcohol (PVA), polyacrylamide, 2-hydroxyethyl methacrylate (HEMA), carboxymethyl cellulose (CMC), polyvinylpyrrolidone (PVP), poly (N-isopropylacrylamide (PNIPAM), chitosan, acrylate-modified PEG, acrylate-modified hyaluronic acid, and the like.

### Polyzwitterion Compounds (Zwitterionic Polymers)

Polyzwitterion compounds herein can include polymers or copolymers including both anionic and cationic groups thereon and, more specifically, any polymer or copolymer in which the monomers are zwitterions.

Polyzwitterion compounds herein can include, but are not limited to, homopolymers and copolymers including polyphosphobetaines, polysulfobetaines, polycarbobetaines, polyphosphocholines, polytrimethylamine N-oxide, polyectoine, poly (3-(N-(2-(methacryloyloxy)ethyl)-N,N-dimethylammonio)propanesulfonate), poly-N-(carboxymethyl)-N,N-dimethyl-2-(methacryloyloxy)ethanaminium) (PCDME). Polyzwitterion compounds herein can specifically include zwitterionic poly(sulfobetaine methacrylate) (PSBMA). In some embodiments, polyzwitterionic compounds herein can include those with photoreactive groups (including but not limited to benzophenone photoreactive groups).

### Heparin Compounds

Various embodiments herein can include a heparin compound. Heparin is a glycosaminoglycan. Heparin compounds herein can include all molecular weights of heparin, low molecular weight heparin, heparan sulfate, heparan sulfate proteoglycans, sodium heparin, high affinity heparin, low affinity heparin, heparin fragments, heparin derivatives, and the like. Heparin compounds herein can include enoxaparin, dalteparin, tinzaparin, danaparoid. Heparin compounds used herein can also include those modified to include a photoreactive group (photo-heparin). As one example, a photoreactive heparin (shown below, Compound I) can be prepared by reacting heparin with benzoyl-benzoyl-epsilon-aminocaproyl-N-oxysuccinimide in dimethylsulfoxide/carbonate buffer, pH 9.0. The solvent can be evaporated and the photoheparin dialyzed against water and lyophilized, and then dissolved in water at a desired concentration. The product can be referred to as BBA-EAC-heparin (referring to the benzophenone photoreactive group benzoyl benzoic acid, BBA; and the spacer, epsilon aminocaproic acid, EAC). However, some embodiments herein can lack a heparin compound.

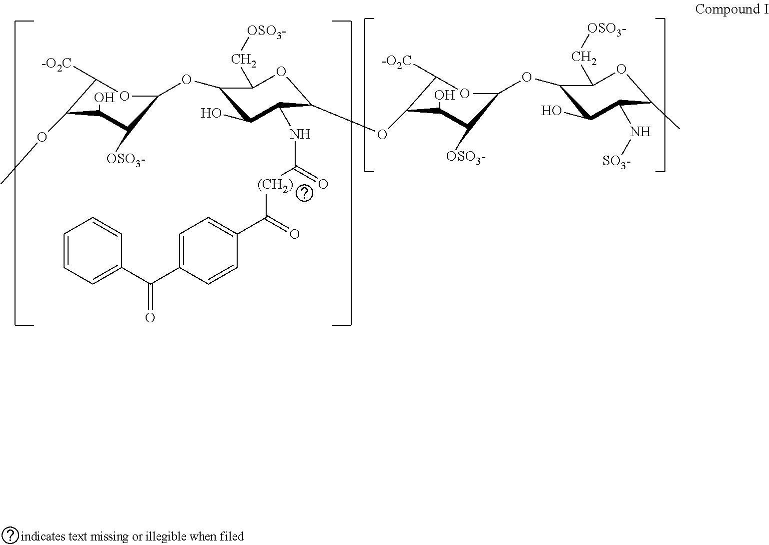

### Polyvinylpyrrolidone Compounds

Various embodiments herein include a polyvinylpyrrolidone homopolymer and/or copolymer. Further details about exemplary polyvinylpyrrolidone polymers (homopolymers and copolymers) are provided as follows. However, it will be appreciated that this is merely provided by way of example and that further variations are contemplated herein.

Polyvinylpyrrolidone polymers herein can include polyvinylpyrrolidone homopolymers as well as polyvinylpyrrolidone subunit containing copolymers. By way of example, polyvinylpyrrolidone copolymers can include subunits of polyvinylpyrrolidone as follows:

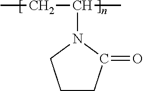

In various embodiments, a PVP copolymer can include at least one including at least one of PVP-co-PEO (polyvinylpyrrolidone-co-polyethylene oxide) and PVP-co-PSB (polyvinylpyrrolidone-co-polysulfobetaine), PVP-co-PAA, and photo-PVP-co-PAA (a photo-derivatized PVP such as PVP modified to include a benzophenone functional group or another photoactivatable functional group).

Polyvinylpyrrolidone polymers herein can be linear or can be branched.

Polyvinylpyrrolidone polymers herein can have various molecular weights such as an average molecular weight from 1 kDa to 3000 kDa. In various embodiments, the polyvinylpyrrolidone polymer has an average molecular weight from 10 kDa to 50 kDa. While not intending to be bound by theory, lower molecular weight PVP polymers can be more effective in PAA complexation. In various embodiments, the polyvinylpyrrolidone polymer has an average molecular weight of less than 15, 12, 10, 8, 6, or 4 kDa, or a molecular weight falling within a range between any of the foregoing. In various embodiments, the polyvinylpyrrolidone polymer has an average molecular weight of between 4 kDa and 12 kDa.

In various embodiments, the polyvinylpyrrolidone component is a blend of different molecular weight PVP compounds. Exemplary non-photo derivatized polyvinylpyrrolidone polymers can include, for example, PVP K12, PVP K30, PVP K90, and the like.

Polyvinylpyrrolidone polymers herein can be used to form a PVP hydrogel.

In various embodiments, the polyvinylpyrrolidone can include a non-photoreactive polyvinylpyrrolidone. However, instead of or in addition to non-photoreactive polyvinylpyrrolidone, in various embodiments, the polyvinylpyrrolidone can also include a photoreactive polyvinylpyrrolidone (“photo-PVP”). Photoreactive polyvinylpyrrolidones can include homopolymers and/or copolymers where they are derivatized to include a photoreactive group. In various embodiments, the photoreactive polyvinylpyrrolidone can specifically include a benzophenone group.

An exemplary photoreactive polyvinylpyrrolidone copolymer can include poly [vinyl pyrrolidone-co-N-(3-(4-benzoylbenzamideo)propyl)methacrylamide] (or PVP-co-APMA with 80 to 99.9 mole percent PVP and 20 to 0.1 mole percent APMA). By way of example, an exemplary photoreactive polyvinylpyrrolidone is as follows:

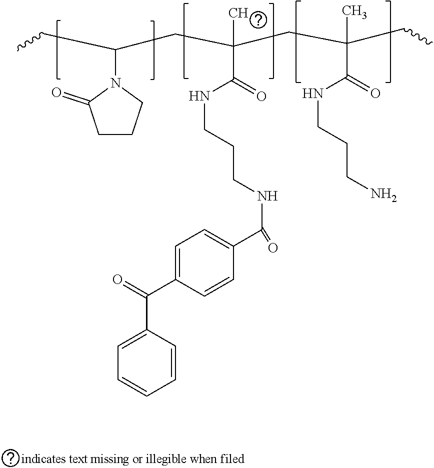

Another exemplary photoreactive polyvinylpyrrolidone copolymer (acetylated PVP-APMA-BBA; or acetylated photo-PVP) is as follows:

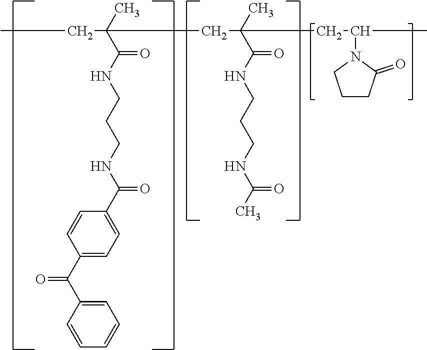

This compound can be prepared by a copolymerization of 1-vinyl-2-pyrrolidone and N-(3-aminopropyl) methacrylamide (APMA), followed by photoderivatization of the polymer using 4-benzoylbenzoyl chloride under Schotten-Baumann conditions. The unreacted amines of the photopolymer can be further acetylated using acetic anhydride.

The polyvinylpyrrolidone used herein can be of various molecular weights. In some embodiments, a non-photoreactive polyvinylpyrrolidone herein can have an average molecular weight from 1 kDa to 3000 kDa. In various embodiments, a non-photoreactive polyvinylpyrrolidone can have an average molecular weight from 10 kDa to 50 kDa. In various embodiments, a non-photoreactive polyvinylpyrrolidone can be a blend of different molecular weight PVP compounds. For example, in various embodiments, the second non-photoreactive polyvinylpyrrolidone of the second layer can be a blend of at least two different molecular weight average PVP compositions.

In various embodiments, a weight ratio of a branched PVP to a poly (acrylic acid) in the same layer or a different layer is from 70:100 to 10:90.

In various embodiments, a non-photoreactive polyvinylpyrrolidone of an outer layer or a PAA containing layer can include a blend of branched and unbranched PVP.

### Crosslinking Agents

Various embodiments herein include a crosslinking agent. Further details about exemplary crosslinking agents are provided as follows. However, it will be appreciated that this is merely provided by way of example and that further variations are contemplated herein.

In some embodiments, the crosslinking agent(s) can have a molecular weight of less than about 1500 kDa, but in other embodiments can be larger. In some embodiments the crosslinking agent can have a molecular weight of less than about 1200, 1100, 1000, 900, 800, 700, 600, 500, or 400 or less, or a molecular weight falling within a range between any of the foregoing.

In various embodiments, cross-linking agents include one or more photoreactive groups attached to a linking group. The cross-linking agent (or linking agent) can be represented by the formula Photo1-LG-Photo2, wherein Photo1 and Photo2 independently represent at least one photoreactive group and LG represents a linking group. The term “linking group” as used herein, refers to a segment or group of molecules configured to connect two or more molecule to each another. In some embodiments, the linking group can include a heteroatom. In some embodiments, the linking group lacks a heteroatom. In one embodiment, the linking group includes at least one silicon atom. In another embodiment, the linking group includes at least one phosphorus atom.

In some embodiments, the linking group can be a degradable linking group, which in other embodiments the linking group can be a non-degradable linking group. The term “degradable linking group” as used herein, refers to a moiety configured to connect one molecule to another, wherein the linking group is capable of cleavage under one or more conditions. The term “biodegradable” as used herein, refers to degradation in a biological system, and includes for example, enzymatic degradation or hydrolysis. It should be noted that the term “degradable” as used herein includes both enzymatic and non-enzymatic (or chemical) degradation. It is also understood that hydrolysis can occur in the presence of or without an acid or base. In one embodiment, the linking agent is water soluble. In another embodiment, the linking agent is not water soluble.

In various embodiments the linking group can function as a spacer, for example, to increase the distance between the photoreactive groups of the linking agent. For example, in some instances it may be desirable to provide a spacer to reduce steric hindrance that may result between the photoreactive groups, which could interfere with the ability of the photoreactive groups to form covalent bonds with a support surface, or from serving as a photoinitiator for polymerization. As described herein, it is possible to vary the distance between the photoreactive groups, for example, by increasing or decreasing the spacing between one or more photoreactive groups.

As described herein, one or more photoreactive groups can be bound to a linking group by a degradable or a non-degradable linkage. In various embodiments, the degradable linkage between the photoreactive group and the linking group includes at least one heteroatom, including, but not limited to oxygen, nitrogen, selenium, sulfur or a combination thereof. In one embodiment, a photoreactive group, linking group and heteroatom form an ether (R¹—O—R²), wherein R¹ is a photoreactive group and R² is a linking group. In another embodiment, a photoreactive group, linking group and heteroatom form an amine,

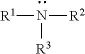

wherein R¹ is a photoreactive group, R² is a linking group, and R³ is hydrogen, aryl or alkyl, a photoreactive group, or a hydroxyl or salt thereof. In one embodiment, R³ is cyclic, linear or branched, saturated or unsaturated, aromatic or heteroaromatic, or a combination thereof. The stability of the ether and/or amine linkage can be influenced depending upon the size (e.g., chain length, branching, bulk, etc.) of the substituents. For example, bulkier substituents will generally result in a more stable linkage (i.e., a linking agent that is slower to degrade in the presence of water and/or acid).

In various embodiments, the linking group includes one or more silicon atoms. In a particular embodiment, the linking group includes one silicon atom (which can be referred to as a monosilane) covalently bound to at least two photoreactive groups. In another embodiment, the linking group includes at least two silicon atoms (which can be referred to as a disilane). In one embodiment, the linking group can be represented by the formula Si—Y—Si, wherein Y represents a linker that can be null (e.g., the linking group includes a direct Si—Si bond), an amine, ether, linear or branched C₁-C₁₀ alkyl, or a combination thereof. In one embodiment, Y is selected from O, CH₂, OCH₂CH₂O and O(CH₂CH₂O)ₙ, wherein n is an integer between 1 and 5, between 1 and 10, between 1 and 15, between 1 and 20, between 1 and 25, or between 1 and 30. One embodiment of a disilane linking agent is shown below

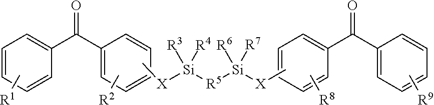

wherein R¹, R², R⁸ and R⁹ can be any substitution, including, but not limited to H, alkyl, halide, hydroxyl, amine, or a combination thereof; R³, R⁴, R⁶ and R⁷ can be alkyl, aryl or a combination thereof; R⁵ can be any substitution, including but not limited to O, alkyl or a combination thereof; and each X, independently, can be O, N, Se, S, or alkyl, or a combination thereof. One specific embodiment is shown below:

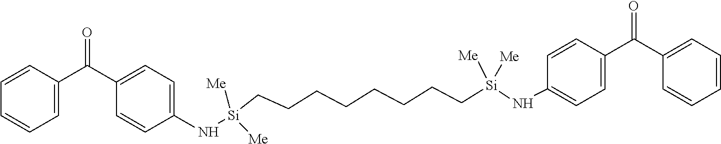 [SMILES: C[Si](C)(CCCCCCCC[Si](C)(C)Nc1ccc(C(=O)c2ccccc2)cc1)Nc1ccc(C(=O)c2ccccc2)cc1]

In various embodiments, the linking agent can be represented by the formula

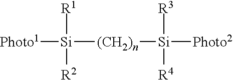

wherein Photo1 and Photo2, independently, represent one or more photoreactive groups and n is an integer between 1 and 10, wherein the linking agent comprises a covalent linkage between at least one photoreactive group and the linking group, wherein the covalent linkage between at least one photoreactive group and the linking group is interrupted by at least one heteroatom. In general, a longer hydrocarbon chain between the two silicon atoms will tend to increase the flexibility of the linking agent and may facilitate crosslinking between a greater number of polymers than a linking agent with a shorter carbon chain, since the photoreactive groups can react with polymers located farther apart from one another. In the formula shown above, R¹, R², R³, R⁴ are independently alkyl or aryl, including, but not limited to cyclic, linear or branched, saturated or unsaturated, aromatic or heteroaromatic, or a combination thereof. In a more particular embodiment, R¹-R⁴ are independently phenyl, methyl, ethyl, isopropyl, t-butyl, or a combination thereof. In another embodiment, R¹-R⁴ can also be, independently, a photoreactive group. In yet another embodiment, R¹-R⁴ can also be, independently, hydroxyl or salt thereof. In one embodiment, the hydroxyl salt includes a counterion that is lithium, sodium, potassium, or a combination thereof.

In another embodiment, the linking agent can be represented by the formula

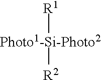

wherein Photo1 and Photo2, independently, represent one or more photoreactive group, wherein the linking agent comprises a covalent linkage between at least one photoreactive group and the linking group, wherein the covalent linkage between at least one photoreactive group and the linking group is interrupted by at least one heteroatom; R¹ and R² are independently alkyl or aryl, including, but not limited to cyclic, linear or branched, saturated or unsaturated, aromatic or heteroaromatic, or a combination thereof. In a more particular embodiment, R¹ and R² are independently phenyl, methyl, ethyl, isopropyl, t-butyl, or a combination thereof. R¹ and R² can also be, independently, a photoreactive group, wherein the linking agent comprises a covalent linkage between at least one photoreactive group and the linking group, wherein the covalent linkage between at least one photoreactive group and the linking group is interrupted by at least one heteroatom; or hydroxyl or salt thereof. In one embodiment, the hydroxyl salt includes a counterion that is lithium, sodium, potassium, or a combination thereof. One embodiment of a monosilane linking agent is shown below

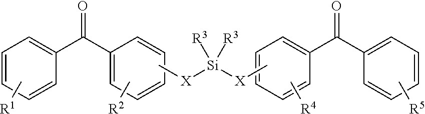

in which R¹ and R⁵ can be any substitution, including, but not limited to H, halogen, amine, hydroxyl, alkyl, or a combination thereof; R² and R⁴ can be any substitution, except OH, including, but not limited to H, alkyl or a combination thereof; R³ can be alkyl, aryl or a combination thereof, including, for example, methyl, ethyl, propyl, isopropyl and butyl; and X, independently, can be O, N, Se, S, alkyl or a combination thereof.

In another embodiment, the linking group includes one or more phosphorous atoms. In one embodiment, the linking group includes one phosphorus atom (which can also be referred to as a mono-phosphorus linking group). In another embodiment, the linking agent includes two phosphorus atoms (which can also be referred to as a bis-phosphorus linking group). In one embodiment, the linking group comprises at least one phosphorus atom with a phosphorus-oxygen double bond (P—O), wherein at least one or two photoreactive groups are bound to the phosphorus atom. In another embodiment, the linking group comprises one phosphorus atom with a phosphorus-oxygen double bond (P═O), wherein two or three photoreactive groups are covalently bound to the phosphorus atom. In another embodiment, the linking group comprises at least two phosphorus atoms, wherein at least one phosphorus atom includes a phosphorus-oxygen double bond (P═O), and at least one or two photoreactive groups are covalently bound to each phosphorus atom.

In a more particular embodiment, the linking agent can be represented by the formula:

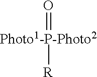

wherein Photo1 and Photo2, independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group, hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In a more particular embodiment, R is phenyl, methyl, ethyl, isopropyl, t-butyl, or a combination thereof.

In another embodiment, the linking agent can be represented by formula:

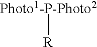

wherein Photo1 and Photo2 independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group (wherein the covalent linkage between the photoreactive group and the linking group may be interrupted by at least one heteroatom), hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In one embodiment, Ris phenyl, methyl, ethyl, isopropyl, t-butyl, or a combination thereof.

In another embodiment, the linking agent can be represented by the formula:

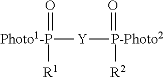

wherein Photo1 and Photo2, independently, represent one or more photoreactive groups, wherein the linking agent comprises a covalent linkage between at least one photoreactive group and the linking group, wherein the covalent linkage between at least one photoreactive group and the linking group is interrupted by at least one heteroatom; Y represents a linker that can be null (i.e., not present, such that the linking group includes a direct P—P bond), N or O, linear or branched C₁-C₁₀ alkyl, or a combination thereof; and R¹ and R² are independently alkyl, aryl, a photoreactive group (wherein the covalent linkage between the photoreactive group and the linking group can be interrupted by at least one heteroatom), hydroxyl or salt thereof, or a combination thereof. In one embodiment, Y is selected from O, CH₂, OCH₂O, OCH₂CH₂O and O (CH₂CH₂O)ₙ, wherein n is an integer between 1 and 5, between 1 and 10, between 1 and 15, between 1 and 20, between 1 and 25, or between 1 and 30. In one embodiment, the hydroxyl salt counterion is lithium, sodium, potassium, or a combination thereof. In a more particular embodiment, R¹ and R² are independently, cyclic, linear or branched hydrocarbon, saturated or unsaturated, aromatic or heteroaromatic, or a combination thereof. In one embodiment, R¹ and R² are independently phenyl, methyl, ethyl, isopropyl, t-butyl, or a combination thereof. In general, a longer hydrocarbon chain between the two phosphorus atoms will tend to increase the flexibility of the linking agent and may facilitate crosslinking between a greater number of polymers than a linking agent with a shorter carbon chain, since the reactive photoreactive groups can react with polymers located farther apart from one another. In one embodiment, Y can be O, CH₂, OCH₂CH₂O and O(CH₂CH₂O)ₙ wherein n is an integer between 1 and 5, between 1 and 10, between 1 and 15, between 1 and 20, between 1 and 25, or between 1 and 30. One embodiment is shown below

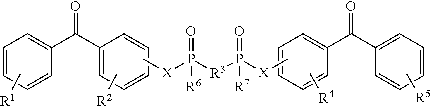

in which R¹, R², R⁴ and R⁵ can be any substitution, including but not limited to H, alkyl, halogen, amine, hydroxyl, or a combination thereof; R³ can be any substitution, including but not limited to O, alkyl, or a combination thereof; and each X can independently be O, N. Se, S, alkyl, or a combination thereof. In one embodiment, the linking agent includes one or more phosphorester bonds and one or more phosphoramide bonds, and can be represented by the formula:

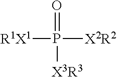

wherein X and X² are, independently, O, N, Se, S or alkyl; R¹ and R² are independently, one or more photoreactive groups, and X³ is O, N, Se, S, alkyl or aryl; R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or a hydroxyl or salt thereof. In one embodiment, the hydroxyl salt counterion is lithium, sodium, potassium, or a combination thereof.

In one embodiment, the linking agent comprises a triphosphorester, which can be represented by the formula.

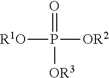

wherein R¹ and R² are independently, one or more photoreactive groups, and R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or hydrogen, or a hydroxyl salt. In one embodiment, the hydroxyl salt counterion is lithium, sodium, potassium, or a combination thereof.

Some specific embodiments include the following linking agents:
(a) bis (4-benzoylphenyl)hydrogen phosphate:

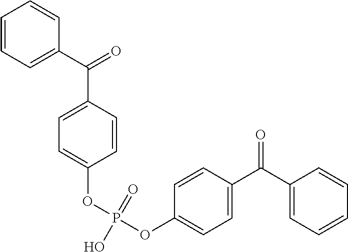 [SMILES: O=C(c1ccccc1)c1ccc(OP(=O)(O)Oc2ccc(C(=O)c3ccccc3)cc2)cc1]

(b) sodium bis (4-benzoylphenyl phosphate):

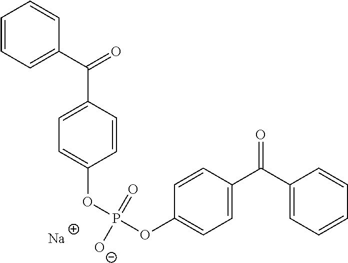 [SMILES: O=C(c1ccccc1)c1ccc(OP(=O)([O-])Oc2ccc(C(=O)c3ccccc3)cc2)cc1 ; [Na+]]

(c) tris (4-benzyolphenyl)phosphate):

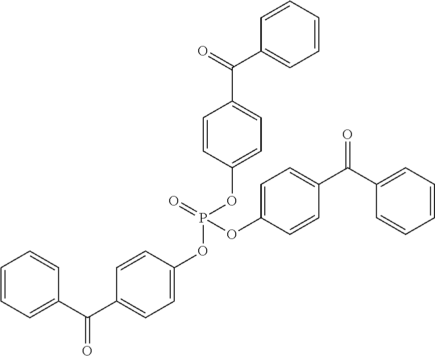 [SMILES: O=C(c1ccccc1)c1ccc(OP(=O)(Oc2ccc(C(=O)c3ccccc3)cc2)Oc2ccc(C(=O)c3ccccc3)cc2)cc1]

(d) tetrakis (4-benzoylphenyl)methylenebis(phosphonate)

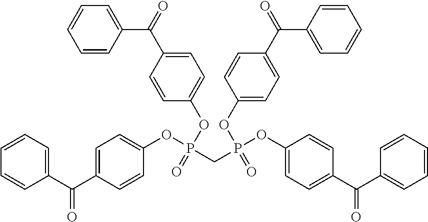 [SMILES: O=C(c1ccccc1)c1ccc(OP(=O)(CP(=O)(Oc2ccc(C(=O)c3ccccc3)cc2)Oc2ccc(C(=O)c3ccccc3)cc2)Oc2ccc(C(=O)c3ccccc3)cc2)cc1]

In another embodiment, the linking agent comprises a triphosphoramide, which can be represented by the formula.

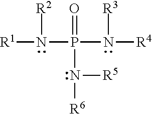

wherein R¹-R⁶ are independently, a photoreactive group, a hydroxyl or salt thereof, alkyl or aryl, or a combination thereof, wherein at least two of R¹-R⁶ are, independently, a photoreactive group. In one embodiment, the hydroxyl salt counterion is lithium, sodium, potassium, or a combination thereof. In a more particular embodiment, R¹-R⁶ are independently cyclic, linear or branched, saturated or unsaturated, aromatic or heteroaromatic, or a combination thereof. In a more particular embodiment, R¹-R⁶ are, independently, phenyl, methyl, ethyl, isopropyl, t-butyl, or a combination thereof.

In some embodiments, the photoactivatable cross-linking agent can be ionic, and can have good solubility in an aqueous composition, such as the first and/or second coating composition. Thus, in some embodiments, at least one ionic photoactivatable cross-linking agent is used to form the coating. In some cases, an ionic photoactivatable cross-linking agent can crosslink the polymers within the second coating layer which can also improve the durability of the coating.

Any suitable ionic photoactivatable cross-linking agent can be used. In some embodiments, the ionic photoactivatable cross-linking agent is a compound of formula I: X₁-Y-X₂ where Y is a radical containing at least one acidic group, basic group, or a salt of an acidic group or basic group. X₁ and X₂ are each independently a radical containing a latent photoreactive group. The photoreactive groups can be the same as those described herein. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X₁ and X₂ can contain photoreactive groups such as aryl ketones. Such compounds include 4,5-bis (4-benzoylphenylmethyleneoxy)benzene-1,3-disulfonic acid or salt; 2,5-bis (4-benzoylphenylmethyleneoxy)benzene-1,4-disulfonic acid or salt; 2,5-bis (4-benzoylmethyleneoxy)benzene-1-sulfonic acid or salt; N,N-bis [2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X₁ and X₂ can contain photoreactive groups that include aryl ketones. Such photoactivatable cross-linking agents include ethylenebis (4-benzoylbenzyldimethylammonium) salt; hexamethylenebis (4-benzoylbenzyldimethylammonium) salt; 1,4-bis (4-benzoylbenzyl)-1,4-dimethylpiperazinediium) salt, bis (4-benzoylbenzyl) hexamethylenetetraminediium salt, bis [2-(4-benzoylbenzyldimethylammonio)ethyl]-4-benzoylbenzylmethylammonium salt; 4,4-bis (4-benzoylbenzyl) morpholinium salt; ethylenebis [(2-(4-benzoylbenzyldimethylammonio)ethyl)-4-benzoylbenzylmethylammonium] salt; and 1,1,4,4-tetrakis (4-benzoylbenzyl) piperzinediium salt. See U.S. Pat. No. 5,714,360. The counter ion is typically a carboxylate ion or a halide. On one embodiment, the halide is bromide.

In other embodiments, the ionic photoactivatable cross-linking agent can be a compound having the formula:

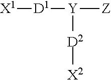

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable cross-linking agents are described in US Patent Application Publication US 2011/0144373 (Swan et al., “Water Soluble Degradable Crosslinker”), the disclosure of which is incorporated herein by reference.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable cross-linking agent can be represented by the formula:

PG²-LE²-X-LE¹-PG¹
wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable cross-linking agents can include, for example, those described in U.S. Provisional Application 61/494,724 filed Jun. 8, 2011 (now U.S. application Ser. No. 13/490,994) (Swan et al., “Photo-Vinyl Primers/Crosslinkers”), the disclosure of which is incorporated herein by reference. Exemplary cross-linking agents can include non-ionic photoactivatable cross-linking agents having the general formula R¹-X-R², wherein R¹ is a radical comprising a vinyl group, X is a radical comprising from about one to about twenty carbon atoms, and R² is a radical comprising a photoreactive group.

Some suitable cross-linking agents are those formed by a mixture of the chemical backbone molecule (such as pentaerythritol) and an excess of a derivative of the photoreactive group (such as 4-bromomethylbenzophenone). An exemplary product is tetrakis (4-benzoylbenzyl ether) of pentaerythritol (tetrakis (4-benzoylphenylmethoxymethyl) methane). See U.S. Pat. Nos. 5,414,075 and 5,637,460.

A single photoactivatable cross-linking agent or any combination of photoactivatable cross-linking agents can be used in forming the coating. In some embodiments, at least one nonionic cross-linking agent such as tetrakis (4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis (4-benzoylbenzyldimethylammonium) salt or at least one anionic photoactivatable cross-linking agent such as 4,5-bis (4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

An exemplary cross-linking agent is disodium 4,5-bis [(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

A further exemplary cross-linking agent is ethylenebis (4-benzoylbenzyldimethylammonium)dibromide. This agent can be prepared as described in U.S. Pat. No. 5,714,360, the content of which is herein incorporated by reference.

Further cross-linking agents can include the cross-linking agents described in U.S. Publ. Pat. App. No. 2010/0274012 and U.S. Pat. No. 7,772,393 the content of all of which is herein incorporated by reference.

In some embodiments, cross-linking agents can include boron-containing linking agents including, but not limited to, the boron-containing linking agents disclosed in U.S. 61/666,516, entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

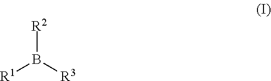

wherein R¹ is a radical comprising a photoreactive group; R² is selected from OH and a radical comprising a photoreactive group, an akyl group and an aryl group; and R³ is selected from OH and a radical comprising a photoreactive group. In some embodiments the bonds B—R¹, B—R² and B—R³ can be chosen independently to be interrupted by a heteroatom, such as O, N, S, or mixtures thereof.

Additional agents for use with embodiments herein can include stilbene-based reactive compounds including, but not limited to, those disclosed in U.S. 61/736,436, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, cross-linking agents, hydrophilic coatings, and associated reagents are disclosed in US2011/0059874; US 2011/0046255; and US 2010/0198168, the content of all of which is herein incorporated by reference. Further exemplary cross-linking agents are described in U.S. Publ. Pat. App. No. 2011/0245367, the content of which is herein incorporated by reference in its entirety.

### Substrates

The substrate can be formed from any desirable material, or combination of materials, suitable for use within the body. In some embodiments the substrate is formed from compliant and flexible materials, such as elastomers (polymers with elastic properties). Exemplary elastomers can be formed from various polymers including polyurethanes and polyurethane copolymers, polyethylene, styrene-butadiene copolymers, polyisoprene, isobutylene-isoprene copolymers (butyl rubber), including halogenated butyl rubber, butadiene-styrene-acrylonitrile copolymers, silicone polymers, fluorosilicone polymers, polycarbonates, polyamides, polyesters, polyvinyl chloride, polyether-polyester copolymers, polyether-polyamide copolymers, and the like. The substrate can be made of a single elastomeric material, or a combination of materials.

Other materials for the substrate can include those formed of polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

Beyond polymers, and depending on the type of device, the substrate can also be formed of other inorganic materials such as metals (including metal foils and metal alloys), glass and ceramics.

Processes to modify substrates described above can include chemical modifications to improve performance characteristics of the substrate. Specific chemical processes that can be used include ozone treatment, chemical oxidation, acid chemical etching, base chemical etching, plasma treatment and corona treatment, surface grafting, thermally activated coating processes (both covalent and non-covalent) and surface modifications including coatings containing dopamine, tannic acid, plant polyphenols and other catechols or catechol containing derivatives of hydrophilic moieties. Additionally, processes to form substrates described above can include physical modifications for example, but not limited to, sand blasting and surface texturing (for example either during or after the molding process of polymers).

In some embodiments, the modification of substrates as described herein can allow for omission of a base coating layer (such as a hydrophilic layer) as substrate surfaces that have been modified will allow for improved adhesion of a hydrophobic therapeutic agent and cationic agent compared with that of a hydrophilic layer.

### Medical Devices

It will be appreciated that embodiments herein include, and can be used in conjunction with, various types of medical devices including, but not limited to, various types of catheters, drug delivery devices such as drug eluting balloon catheters, drug-containing balloon catheters, stents, grafts, and the like.

Some embodiments described herein can be used in conjunction with balloon expandable flow diverters, and self-expanding flow diverters. Other embodiments can include uses in contact with angioplasty balloons (for example, but not limited to, percutaneous transluminal coronary angioplasty and percutaneous transluminal angioplasty). Yet other embodiments can include uses in conjunction with sinoplasty balloons for ENT treatments, urethral balloons and urethral stents for urological treatments and gastro-intestinal treatments (for example, devices used for colonoscopy). Hydrophobic active agent can be transferred to tissue from a balloon-like inflatable device or from a patch-like device. Other embodiments of the present disclosure can further be used in conjunction with micro-infusion catheter devices. In some embodiments, micro-infusion catheter devices can be used to target active agents to the renal sympathetic nerves to treat, for example, hypertension.

Other exemplary medical applications wherein embodiments of the present disclosure can be used further encompass treatments for bladder neck stenosis (e.g. subsequent to transurethral resection of the prostrate), laryngotrachial stenosis (e.g. in conjunction with serial endoscopic dilatation to treat subglottic stenosis, treatment of oral cancers and cold sores and bile duct stenosis (e.g. subsequent to pancreatic, hepatocellular of bile duct cancer). By way of further example, embodiments herein can be used in conjunction with drug applicators. Drug applicators can include those for use with various procedures, including surgical procedures, wherein active agents need to be applied to specific tissue locations. Examples can include, but are not limited to, drug applicators that can be used in orthopedic surgery in order to apply active agents to specific surfaces of bone, cartilage, ligaments, or other tissue through physical contact of the drug applicator with those tissues. Drug applicators can include, without limitation, hand-held drug applicators, drug patches, drug stamps, drug application disks, and the like.

### OTHER EMBODIMENTS

In various embodiment, a coated medical device is included having a substrate and a coating disposed thereon. The coating can include a degradable polymer. The degradable polymer can include a polymeric backbone with degradable linkages and polymeric chains grafted onto the backbone. The chains can include a polyzwitterionic polymer, polyethylene glycol, or a copolymer thereof.

In various embodiments, the polymeric backbone can include a multi-block copolymer with subunits selected from the group consisting of lactide, glycolide, e-caprolactone, and polyethylene glycol.

In an embodiment, the coated medical device can further include a base coat, wherein the base coat is disposed between the substrate and the coating. In an embodiment, the base coat can include a tie layer.

In an embodiment, the chains can be covalently bonded to the polymeric backbone with the reaction product of a photoreactive compound. In an embodiment, the photoreactive compound can include a benzophenone moiety.

In various embodiments, the coating is anti-fouling. In various embodiments, the coating is lubricious.

In an embodiment, the coated medical device is an implantable medical device. In an embodiment, the coated medical device is a transitorily implantable medical device. In an embodiment, the coated medical device is a chronically implantable medical device.

In an embodiment, a coated medical device is included having a substrate and a coating disposed on the substrate. The coating can include a degradable polymer layer. The degradable polymer layer can include a multi-block copolymer with subunits selected from the group consisting of lactide, glycolide, e-caprolactone, and polyethylene glycol. The coating can also include a top layer. A photo-PVP (a photo-derivatized PVP polymer or copolymer), a heparin compound, and a polyacrylamide polymer.

In various embodiment, the top layer further can include a cross-linking compound. In an embodiment, the cross-linking compound can include sodium bis (4-benzoylphenyl)phosphate.

In an embodiment, the photo-PVP can include poly [vinyl pyrrolidone-co-N-(3-(4-benzoylbenzamideo) propyl) methacrylamide]. In an embodiment, the polyacrylamide polymer can include poly [acrylamide-co-sodium-2-acrylamido-2-methylpropanesulfonate-co-N-(3-(4-benzoylbenzamido) propyl) methacrylamide] or N-Acetylated poly [acrylamide-co-sodium-2-acrylamido-2-methylpropanesulfonate-co-N-(3-(4-benzoylbenzamido) propyl) methacrylamide]-co-methoxy poly(ethylene glycol).

In an embodiment, a coated medical device is included having a substrate and a coating disposed on the substrate. The coating can include a degradable polymer. The degradable polymer can include a polymeric backbone with degradable linkages. The polymeric backbone can include a multi-block copolymer with subunits selected from the group consisting of lactide, glycolide, e-caprolactone, and polyethylene glycol, and subunits including at least one selected from the group consisting of sebacic acid and glycerol.

Aspects may be better understood with reference to the following examples. These examples are intended to be representative of specific embodiments, but are not intended as limiting the overall scope of embodiments herein.

### EXAMPLES

### Example 1: Formation of Coatings

Coating compositions were formed by mixing components together in a solvent of isopropyl alcohol and water resulting in compositions having the following components:

### Heparin Control:

1. 4 mg/ml Photo-Heparin
2. 13 mg/ml Photo-PVP
3. 5 mg/ml PVP K90

### Test Composition 1:

1. 4 mg/ml Photo-Heparin
2. 5, 11, or 18 mg/ml-85/15 by weight (PSBMA/Photo-PVP)

### Test Composition 2:

1. 4 mg/ml Photo-Heparin
2. 5, 11, or 18 mg/ml-95/4/1 by weight (PSBMA/PVP K90/Photo-PVP)

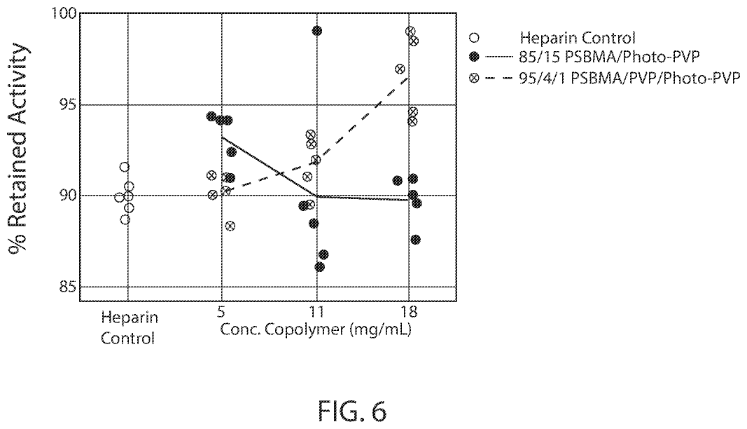

The coating compositions were applied onto a device surface and then the resulting coatings were assessed for heparin activity retention. The results are shown in FIG. 6 showing percent retained heparin (anti-thrombogenic) activity. This example shows an effect on heparin activity retention by zwitterion (PSBMA) in combination with PVP K90.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An anti-fouling coated medical device comprising:
a substrate; and
a coating, wherein the coating is disposed on the substrate, the coating comprising
a hydrogel; and
a polyzwitterion, the polyzwitterion comprising
a bound portion; and
an unbound portion; and
a heparin compound.

2. The anti-fouling coated medical device of claim 1, the bound portion comprising a cross-linked portion.

3. The anti-fouling coated medical device of claim 1, the unbound portion comprising an uncross-linked portion.

4. The anti-fouling coated medical device of claim 1, wherein the bound portion remains bound to the hydrogel after exposure to an aqueous environment.

5. The anti-fouling coated medical device of claim 1, the hydrogel comprising a PVP polymer or copolymer.

6. The anti-fouling coated medical device of claim 5, wherein the PVP polymer or copolymer is not covalently bound into the coating.

7. The anti-fouling coated medical device of claim 5, wherein a portion of the PVP polymer or copolymer is configured to elute off the coating after implant in a patient.

8. The anti-fouling coated medical device of claim 5, the PVP polymer or copolymer comprising:
a photoreactive PVP polymer or copolymer; and
a non-photoreactive PVP polymer or copolymer.

9. The anti-fouling coated medical device of claim 1, the heparin compound comprising a photo-heparin.

10. The anti-fouling coated medical device of claim 1, wherein the bound portion of the polyzwitterion is covalently bound to the hydrogel.

11. (canceled)

12. The anti-fouling coated medical device of claim 1, wherein the bound portion of the polyzwitterion is covalently bound to the hydrogel via the reaction product of a photoactivatable group.

13. The anti-fouling coated medical device of claim 1, the polyzwitterion further comprising a photo reactive polyzwitterion.

14. The anti-fouling coated medical device of claim 1, the polyzwitterion further comprising a betaine compound.

15. The anti-fouling coated medical device of claim 1, the polyzwitterion further comprising at least one selected from the group consisting of a phosphobetaine methacrylate, a sulfobetaine methacrylate, and a carboxybetaine methacrylate.

16. The anti-fouling coated medical device of claim 1, the polyzwitterion further comprising a poly (sulfobetaine methacrylate).

17. (canceled)

18. The anti-fouling coated medical device of claim 1, the coating further comprising an anti-platelet component.

19. The anti-fouling coated medical device of claim 18, the anti-platelet component comprising at least one selected from the group consisting of an elutable macromer and a non-prodrug anti-platelet agent.

20. The anti-fouling coated medical device of claim 19, the elutable macromer comprising at least one selected from the group consisting of a Pluronic polymer, a PVP polymer, a polyethylene glycol polymer, and a polyethylene oxide polymer.

21. The anti-fouling coated medical device of claim 19, the non-prodrug anti-platelet agent comprising at least one selected from the group consisting of Cangrelor, II (Original) A platelet inhibitor, and IIB platelet inhibitor.

22. The anti-fouling coated medical device of claim 1, the hydrogel comprising a polyacrylic acid polymer.

23-79. (canceled)
